(19) United States
(12) Patent Application Publication
(10) Pub. No.: US 2009/0029481 A1
(43) Pub. Date: Jan. 29, 2009

(54) Method for separating target component using magnetic nanoparticles

(76) Inventors: Masayoshi Kojima, Minami-ashigara-shi (JP); Hiroyuki Hirai, Minami-ashigara-shi (JP); Shigeki Kageyama, Minami-ashigara-shi (JP); Yoshihiko Abe, Minami-ashigara-shi (JP)

Correspondence Address: BIRCH STEWART KOLASCH & BIRCH, PO BOX 747, FALLS CHURCH, VA, 22040-0747, US

(21) Appl. No.: 11/919,869

(22) PCT Filed: Jun. 29, 2006
(86) PCT No.: PCT/JP2006/313442

§ 371 Date: Nov. 5, 2007

(30) Foreign Application Priority Data
Jun. 30, 2005 (JP) 2005-191267
May 30, 2006 (JP) 2006-149257

(51) Int. Cl. G01N 33/48; B01J 19/08

(52) U.S. Cl. 436/501; 422/68.1

(57) ABSTRACT

An object to be achieved by the present invention is to provide a method for determining the amount of a component contained in a specific lipoprotein fraction in a biological sample using an automatic analyzer, without the requirement of a step of fractionating a sample by centrifugation. The present invention provides a method for separating a target component in a biological sample, which comprises the steps of: (1) causing a biological sample to come into contact with independently dispersed magnetic nanoparticles having a particle size of 50 nm or less, which have anionic functional groups on their surfaces, so as to form an agglutinate of the magnetic nanoparticles and biomolecules capable of interacting with the magnetic nanoparticles; and (2) collecting the agglutinate by an external magnetic field.

### TECHNICAL FIELD

The present invention relates to a method for separating a target component from a biological sample such as serum or blood plasma in the field of life science, medical diagnosis, or the like. For example, the present invention relates to a method for separating a specific lipoprotein fraction in a biological sample such as serum or blood plasma. The method of the present invention can be used for determination of the amount of cholesterol or the like in an HDL fraction, where such determination is performed for use in clinical examination.

### BACKGROUND ART

In blood, lipids bind to apoproteins to form lipoproteins, and then the lipoproteins are metabolized. Lipoproteins can be classified in terms of specific gravity into fractions such as those of chylomicrons (CMs), very low density lipoproteins (VLDLs), intermediate density lipoproteins (IDLs), low density lipoproteins (LDLs), and high density lipoproteins (HDLs). It is known that various diseases affect the metabolism of these lipoproteins so that lipoprotein fractions increase or decrease in blood. In particular, it is known that HDLs receive cholesterol from various tissues including arterial wall and are involved in removing action of cholesterols accumulated in cells, and that HDLs are a risk prevention factor for various arteriosclerosis such as coronary arteriosclerosis, and its level in blood is a useful indicator for foreseeing the onset of arteriosclerosis. Therefore, measurement of cholesterol amount in an HDL fraction is performed in clinical examinations for preventing or diagnosing ischemic heart disease and the like.

As methods for fractionating lipoproteins, an ultracentrifugation method, an electrophoresis method, a gel filtration method, and the like are known. Because of very complicated procedures required for these methods, these methods are used infrequently in clinical examinations. Thus, a precipitation method (fraction method) is often used in clinical examinations. A method which is generally and commonly used as a method for determining HDL cholesterol (hereinafter referred to as HDL-C) is a fractionating method which involves agglutinating lipoprotein other than HDL by adding fractionating agent to a sample, removing the lipoprotein by centrifugation, and then measuring cholesterol in the supernatant containing only the separated HDL. In order to determine the amount of cholesterol in an HDL fraction, the amount of cholesterol contained in an HDL fraction of a collected supernatant can be measured using a known reagent for determination of cholesterol amount.

As a precipitating agent that is used in the above-described precipitation method, a polyanion or a combination of a polyanion and a divalent cation is often used. Known examples of such polyanion include sulfated polysaccharides such as dextran sulfate and heparin, phosphotungstic acid and salts thereof, and polyethylene glycol. Known examples of a divalent cation include Mg²⁺, Mn²⁺, Ca²⁺, and Ni²⁺.

However, since the above-mentioned precipitation method involves an operation for separation by adding a fractionating agent, it is problematic in that relatively large amount of a sample is necessary, an instrument such as centrifuge is necessary, and an error of artificial operation is likely to occur. Further, the precipitation (fraction) method is problematic when it is applied to an automatic analyzer that is often used in clinical examinations. Specifically, the precipitation method requires a step of fractionating a sample by centrifugation, so that the method requires a specific treatment time for obtaining a fraction of an analysis subject. Hence, rapid analysis of large amounts of samples is difficult with this method.

Recently, a direct method which does not require these complicated operation and can be set in an automatic analyzer, has been rapidly spread. For example, a method is known which involves fully reacting lipoprotein other than HDL with a cyclodextrin sulfate which is used as an agglutinating agent, and then allowing an enzyme which was modified with polyethyleneglycol to act thereon, so as to specifically measure cholesterol in HDL. However, it was necessary to modify cyclodextrin in order to suppress the reaction of co-existing lipoproteins other than HDL, and to use high cost products such as enzyme and antibody.

Furthermore, an assay method has been reported whereby the precipitation method is performed with the use of dry slides. However, a problem arises in that the configuration of such slides becomes complicated (JP Patent Publication (Kokai) No. 2005-49346 A). Further, also in the field of the dry chemistry, the precipitation method has been mainly used, but recently a new technique of dry type test piece using a direct method has been devised. However, such technique involves very complicated steps in the production process and is problematic in high cost.

JP Patent Publication (Kokai) No. 6-242110 A (1994) discloses that the amount of a component contained in a specific lipoprotein fraction in a biological sample is directly determined by: agglutinating lipoprotein fractions other than the specific fraction of interest; causing the component to react with a reagent with which the component the amount of which is to be determined can be detected; simultaneously with or after the termination of the reaction, dissolving the agglutinated fractions; and then measuring changes resulting from the reaction.

Furthermore, JP Patent No. 2913608 discloses a method for separating lipoproteins of a first class in a sample from lipoproteins of a second class in such sample. Specifically, the method involves precipitating lipoproteins of the second class using a reagent for selective chemical precipitation, causing the sample to come into contact with magnetically reactive particles (where the magnetically reactive particles induce sedimentation of lipoproteins that have been precipitated upon the sedimentation of the particles), placing the sample within a magnetic field until the magnetically reactive particles are sedimented, so as to sediment the precipitated lipoproteins of the second class, and then allowing the lipoproteins of the first class to remain in the supernatant of the sample. In the case of this method, addition of a precipitating agent such as dextran sulfate and magnetic particles to lipoproteins causes the precipitation of fractions other than a specific lipoprotein fraction. Next, when magnet is caused to act on the resultant, magnetic particles and the precipitate form a mixture. The magnetic particles and the precipitate are precipitated together and separated. Such a magnetic particle is a magnetic body having no reactivity with lipoproteins. Finally, the amount of cholesterol in the specific fraction that has remained in the supernatant is determined.

On the other hand, a method for separation using magnetic particles has been used, and some products are commercially available (Iatron Co. and OrthoClinical). However, since these methods use magnetic particles having a size of 100 nm or more, it was necessary to stir it immediately before use and confirm that it was fully uniformized.

### DISCLOSURE OF THE INVENTION

An object to be achieved by the present invention is to address the above described problems in the prior art. Specifically, an object to be achieved by the present invention is to provide a method for determining the amount of a component contained in a specific lipoprotein fraction in a biological sample using an automatic analyzer, without the requirement of a step of fractionating a sample by centrifugation. In particular, an object to be achieved by the present invention is to provide a useful method for determining the amount of cholesterol in an HDL fraction.

As a result of intensive studies to achieve the above objects, the present inventors have discovered that magnetic nanoparticles can cause agglutination of lipoprotein fractions other than a specific lipoprotein fraction so as to allow the easy separation of such lipoprotein fractions from the specific lipoprotein fraction with the use of a magnetic field. The present inventors have further revealed that the amount of a target component can be precisely determined by detecting the component contained in a specific fraction that has remained in a supernatant. Thus, the present inventors have completed the present invention.

Thus, the present invention provides a method for separating a target component in a biological sample, which comprises the steps of: (1) causing a biological sample to come into contact with independently dispersed magnetic nanoparticles having a particle size of 50 nm or less, which have anionic functional groups on their surfaces, so as to form an agglutinate of the magnetic nanoparticles and biomolecules capable of interacting with the magnetic nanoparticles; and (2) collecting the agglutinate by an external magnetic field.

Preferably, the biomolecules capable of interacting with the magnetic nanoparticles have a size that is the same as or greater than the particle size of the magnetic nanoparticles.

Preferably, the magnetic nanoparticles are surfaces-modified with a compound represented by the formula R¹—(OCH₂CH₂)ₙ—O-L-X wherein R¹ represents a C1-24 alkyl group, n represents an integer of 1 to 20, L represents a single bond or a C1-10 alkylene group, and X represents a carboxylic acid group, a phosphoric acid group, a sulfonic acid group, or a boric acid group.

Preferably, an agglutinate of lipoproteins other than a specific lipoprotein fraction in a biological sample and magnetic nanoparticles is formed, and the agglutinate is collected by an external magnetic field, so as to separate the specific lipoprotein fraction in the biological sample.

Preferably, the specific fraction is a high density lipoprotein (HDL).

Preferably, the specific fraction is separated for the determination of the amount of cholesterol contained in the specific fraction.

Preferably, the biological sample is caused to come into contact with magnetic nanoparticles in the coexistence of an agglutination-promoting agent.

Preferably, a polyanion is used as the agglutination-promoting agent.

Preferably, the polyanion is a polyanion which is selected from phosphotungstic acid, dextrin sulfate, cyclodextrin sulfate, Calixarene, or heparin.

Preferably, the independently dispersed magnetic nanoparticles having a particle size of 50 nm or less are magnetites.

Another aspect of the present invention provides a clinical examination method, which comprises the steps of (1) separating a target component in a biological sample by the aforementioned method according to the present invention, and (2) determining the amount of the thus separated target component.

Preferably, the amount of a target component is determined using a dry analytical element.

Further another aspect of the present invention provides an automatic clinical examination apparatus, which comprises at least (1) a vessel wherein magnetic nanoparticles are caused to come into contact with a biological sample so as to form an agglutinate, (2) a magnetic field generation means for generating a magnetic field for collecting the agglutinate within the vessel, and (3) a dry analytical element for detecting a target component in the biological sample which was separated from the agglutinate.

Further another aspect of the present invention provides an examination kit for performing the aforementioned method according to the present invention, which comprises independently dispersed magnetic nanoparticles having a particle size of 50 nm or less which have anionic functional groups on their surfaces.

### BRIEF DESCRIPTION OF THE DRAWINGS

FIG. 1 shows the result of the measurement with a fraction solution without fraction assistant agent.

FIG. 2 shows the result of the measurement with a fraction solution with dextrin sulfate as a fraction assistant agent.

### PREFERRED EMBODIMENTS OF THE INVENTION

The embodiments of the present invention will be described in detail as follows.

The method for separating a target component in a biological sample according to the present invention comprises the following steps of:

(1) causing a biological sample to come into contact with independently dispersed magnetic nanoparticles having a particle size of 50 nm or less, which have anionic functional groups on their surfaces, so as to form an agglutinate of the magnetic nanoparticles and biomolecules capable of interacting with the magnetic nanoparticles; and
(2) collecting the agglutinate by an external magnetic field.

By means of the use of the method of the present invention, fractions other than a specific lipoprotein fraction in blood can be captured an agglutinated by magnetic nanoparticles having anionic functional groups such as carboxylic acid on their surfaces. These agglutinates can be separated by magnets. Subsequently, the amount of cholesterol in the specific fraction that has remained in the supernatant can be determined.

According to the method of the present invention, the amount of a component contained in a specific lipoprotein fraction in a biological sample is rapidly determined as follows. Lipoprotein fractions other than the specific fraction are agglutinated, the resultant is caused to react with a reagent or a slide with which a component (that has remained in the supernatant) the amount of which is to be determined can be detected, and then a product generated by the reaction is measured. Thus, the amount of a component contained in the specific lipoprotein fraction can be determined. Here, a specific fraction may be a high density lipoprotein (HDL). Moreover, a component the amount of which is to be determined may be cholesterol.

In the present invention, independently dispersed magnetic nanoparticles having a particle size of 50 nm or less, which have anionic functional groups on their surfaces, are used to cause agglutination of components (e.g., fractions other than a specific lipoprotein fraction) other than a target component. “Independently dispersed” means a state where particles are independently dispersed without forming any agglutinates in a solution. In addition, magnetic nanoparticles have a particle size of 50 nm or less, further preferably 40 nm or less, and particularly preferably 30 nm or less.

As magnetic nanoparticles, any particles can be used, as long as the particles can be dispersed or suspended in an aqueous medium and can be separated from a dispersion liquid or a suspension through application of a magnetic field. Examples of magnetic nanoparticles that are used in the present invention include: a salt, oxide, boride or sulfide of iron, cobalt or nickel; and rare earth elements having high magnetic susceptibility (e.g., hematite and ferrite). Specific examples of such magnetic nanoparticles that can also be used herein include ferromagnetic ordered alloys such as a magnetite (Fe₃O₄), FePd, FePt, and CoPt. A preferable magnetic nanoparticle in the present invention is selected from metal oxides and particularly from the group consisting of an iron oxide and a ferrite (Fe,M)₃O₄. Examples of such iron oxide particularly include a magnetite, a maghemite, and a mixture thereof. In the above formula, “M” represents a metal ion capable of forming a magnetic metal oxide when it is used in combination with the iron ion. Such metal ion is typically selected from transition metals and is most preferably Zn²⁺, Co²⁺, Mn²⁺, Cu²⁺, Ni²⁺, Mg²⁺, or the like. The molar ratio of M/Fe is determined according to the stoichiometrical composition of a selected ferrite. A metallic salt is supplied in a solid or solution form and is preferably a chloride salt, a bromide salt, or a sulfate. Of these, an iron oxide and a ferrite are preferable in view of safety. A magnetite (Fe₃O₄) is particularly preferable.

Magnetic nanoparticles that are used in the present invention have anionic functional groups on their surfaces. Examples of anionic functional groups include a carboxylic acid group, a phosphoric acid group, a sulfonic acid group, and a boric acid group. In particular, a carboxyl group is preferable.

Preferably, magnetic nanoparticles have a surface which is modified with a compound represented by the formula R¹—(OCH₂CH₂)ₙ—O-L-X, can be used. In the formula, R¹ represents a C1-24 alkyl group, n represents an integer of 1 to 20, L represents a single bond or a C1-10 alkylene group, and X represents a carboxylic acid group, a phosphoric acid group, a sulfonic acid group, or a boric acid group.

In the present invention, magnetic nanoparticles are caused to come into contact with a biological sample. Depending on the biological sample, magnetic nanoparticles can also be caused to come into contact with the biological sample in the presence of an agglutination-promoting agent. Here, an agglutination-promoting agent means a substance that induces agglutination. An appropriate substance can be used alone or appropriate substances can be used in combination, depending on the type of a fraction to be agglutinated. An antibody that is against a fraction other than a specific lipoprotein fraction and causes an immunoagglutination reaction can also be used as an agglutination-promoting agent. Any agglutination-promoting agent can be used, as long as it enables achievement of the purpose of the present invention. It is preferred to add a polycation or polyanion as an agglutination-promoting agent in order to control agglutination speed. For example, for the purpose of causing agglutination of lipoprotein fractions other than an HDL fraction, polyethylene glycol (PEG) as well as polyanion can be used. Phosphotungstic acid, dextrin sulfate, cyclodextrin sulfate, Calixarene, heparin or the like can be used as a polyanion. These can be used alone or can be used in combination with a cation such as Mg²⁺, Mn²⁺, Ca²⁺, Li⁺, or Ni²⁺. When an agglutination-promoting agent is used in the present invention, dextrin sulfate is particularly preferable.

According to a preferred embodiment of the present invention, agglutinates of lipoproteins other than a specific lipoprotein fraction in a biological sample and magnetic nanoparticles are formed. The agglutinates are then collected with the use of an external magnetic field, so that the specific lipoprotein fraction in the biological sample can be separated. “Specific fraction” used herein preferably means a high density lipoprotein (HDL). In the present invention, a specific fraction can be separated in order to determine the amount of cholesterol contained in the specific fraction.

As a reagent that is used for detecting and determining the amount of a component contained in a specific lipoprotein fraction in the present invention, various reagents known in the field of clinical examination or the like can be used. For example, as a reaction for cholesterol amount determination (when the amount of cholesterol in an HDL fraction is determined), an enzyme reaction referred to as an enzyme method with high reaction specificity can be used. Examples of such enzyme method include: a method that involves measuring absorbance in the visible region with the use of cholesterol esterase (CE) and cholesterol oxidase (CO) in combination with peroxidase (POD) and chromogen; and a method that involves measuring absorbance in the ultraviolet region with the use of CE and cholesterol dehydrogenase (CHD) in combination with a coenzyme. Specifically, when the amount of cholesterol in an HDL fraction is determined, a reagent using CE, CO, and POD or a reagent using CE and CHD can be used. Moreover, the amount of cholesterol in an HDL fraction can also be determined using a dry analytical element containing the above reagent.

Furthermore, the present invention provides an automatic clinical examination apparatus, which comprises at least (1) a vessel wherein magnetic nanoparticles are caused to come into contact with a biological sample so as to form an agglutinate, (2) a magnetic field generation means for generating a magnetic field for collecting the agglutinate within the vessel, and (3) a dry analytical element for detecting a target component in the biological sample which was separated from the agglutinate. The type and shape of such vessel in which magnetic nanoparticles are caused to come into contact with a biological sample so as to form an agglutinate are not particularly limited. Such vessel may be a general reaction vessel (including a tube or the like) having at least one opening. A magnet or the like can be used as a magnetic field generation means. Furthermore, a dry analytical element containing a reagent for detecting a target component can be used. When the target component is cholesterol, a combination of CE, CO and POD or a combination of CE and CHD can be contained as reagents. The configuration of a dry analytical element is not particularly limited. For example, a dry analytical element that can be used herein is configured with at least one functional layer and at least one development layer that are layered in this order on one side of a planar water-impermeable support so as to form an integrated laminate. The various above reagents may also be contained in the functional layer and, if necessary, in the development layer.

The present invention will be further described specifically by referring to examples. However, the scope of the present invention is not limited by these examples.

### EXAMPLES

### Example 1

### Preparation of Magnetic Nanoparticle Dispersion Liquid

10.8 g of iron (III) chloride 6-hydrate and 6.4 g of iron (II) chloride 4-hydrate were dissolved in and mixed with 80 ml of a 1N hydrochloric acid aqueous solution. While agitating the solution, 96 ml of ammonia water (28 wt. %) was added to the solution at a rate of 2 ml/minute. After subsequent heating at 80° C. for 30 minutes, the resultant was cooled to room temperature. The thus obtained agglutinate was purified by decantation using water. The generation of magnetites (Fe₃O₄) having a crystallite size of approximately 12 nm was confirmed by an X-ray diffraction method.

100 ml of an aqueous solution (the pH of which had been adjusted at 6.8 using NaOH) prepared by dissolving 2.3 g of polyoxyethylene (4,5) lauryl ether acetate was added to the above-obtained agglutinate for dispersion of the agglutinates. Thus, magnetic nanoparticle dispersion liquid was prepared.

### Example 2

10 μl of phosphotungstic acid (0.2% or 0.8%) was added to 190 μl of a magnetic nanoparticle solution with Fe₃O₄ content of 10.2 g/l. Furthermore, 50 μl of a standard serum (116 mg/dl LDL-C and 86.1 mg/dl HDL-C) was added. The solution was agitated using a vortex mixer and then allowed to stand at room temperature for 30 seconds. The solution was transferred onto a magnet and then allowed to stand for 30 seconds. The supernatant was collected. The amount of cholesterol derived from each lipoprotein was determined using LDL-C-HDL-C detection kit (produced by KYOWA MEDEX CO., LTD.). The results are shown in the Table 1 below. Separation and removal of LDL and capability of determining the amount of HDL could be confirmed.

(1) 0.20% phosphotungstic acid added
(2) 0.80% phosphotungstic acid added

TABLE 1

| | LDL-C | LDL-C | HDL-C | HDL-C |
|---|---|---|---|---|
| | measurement | content | measurement | content |
| (1) | 0 mg/dl | 23.2 mg/dl | 17.9 mg/dl | 17.2 mg/dl |
| (2) | 0 mg/dl | 23.2 mg/dl | 17.0 mg/dl | 17.2 mg/dl |

### Example 3

10 μl of 0.1M MES buffer (pH 6.0) containing 0.5% phosphotungstic acid was added to 190 μl of a magnetic nanoparticle solution with Fe₃O₄ content of 3.62 g/l. 50 μl of a control serum (119 mg/dl LDL-C and 26 mg/dl HDL-C) was added to the solution. The solution was agitated using a vortex mixer and then allowed to stand at room temperature for 30 seconds. The solution was then transferred onto a magnet and then allowed to stand for 30 seconds. The supernatant was collected and then spotted onto FUJI DRY-CHEM HDL-C-P slide (produced by FUJI PHOTO FILM CO., LTD.). The amount of HDL cholesterol was then determined. Furthermore, a supernatant was obtained by fractionation through centrifugation using a fractionation test solution (PR) supplied with the HDL-C-P slides. The supernatant was collected and then spotted onto the slide. Data obtained through determination are also shown.

HDL-C measurement which was determined after treatment with magnetic nanoparticles:

28.06 mg/dl
HDL-C measurement which was determined after treatment with PR:
26.28 mg/dl

As shown in the above results, in the determination of the amount of cholesterol using FUJI DRY-CHEM HDL-C-P slide, the result obtained through fractionation using magnetic nanoparticles was almost the same as that obtained by a conventional method using PR. Moreover, the pretreatment time could be shortened from 20 minutes to 1 minute.

### Example 4

### Preparation of a Fraction Solution without Fraction Assistant Agent

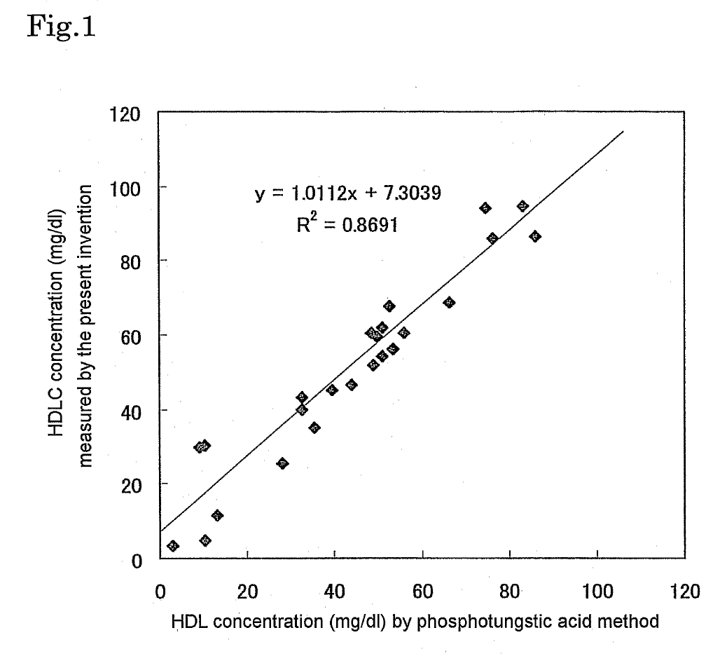

40 μl of a 0.2M MES buffer (pH 5.0) was added to 120 μl of a magnetic nanoparticle (size: 12-15 nm) solution with an Fe₃O₄ content of 15 g/L, so as to prepare a fraction solution. 40 μl of a sample was added to the thus prepared fraction solution, and the solution was agitated. The solution was then allowed to stand for 30 seconds. The vessel containing the mixed solution was then placed on a neodium magnet and then allowed to stand for 60 seconds. The supernatant was collected and then spotted onto FUJI DRY-CHEM HDL-C slide (produced by FUJI PHOTO FILM CO., LTD.). The amount of HDL cholesterol was then determined. For comparison, values obtained by measuring the samples by phosphotungstic acid method were used. The multiple sample correlation of 20 samples (N=20) is shown in FIG. 1.

### Example 5

### Preparation of a Fraction Solution with Dextrin Sulfate as a Fraction Assistant Agent

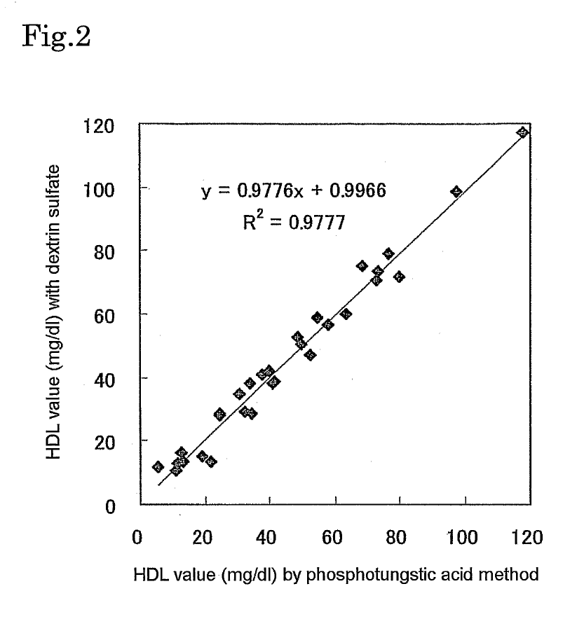

40 μl of a 0.2M MES buffer (pH 5.0) and 10 μl of a 0.4% dextrin sodium sulfate (MW500,000) (Wako Pure Chemical Industries, Ltd.) were added to 100 μl of a magnetic nanoparticle (size: 12-15 nm) solution with an Fe₃O₄ content of 21 g/L, so as to prepare a fraction solution. 50 μl of a sample was added to the thus prepared fraction solution, and the solution was agitated. The solution was then allowed to stand for 30 seconds. The vessel containing the mixed solution was then placed on a neodium magnet and then allowed to stand for 60 seconds. The supernatant was collected and then spotted onto FUJI DRY-CHEM HDL-C slide (produced by FUJI PHOTO FILM CO., LTD.). The amount of HDL cholesterol was then determined. For comparison, values obtained by measuring the samples by phosphotungstic acid method were used. The multiple sample correlation of 20 samples (N=20) is shown in FIG. 2.

### INDUSTRIAL APPLICABILITY

With the method of the present invention, lipoprotein fractions other than a specific lipoprotein fraction can be rapidly separated by a magnetic field after agglutination thereof. Hence, the method of the present invention enables measurement using an automatic analyzer in clinical examination through direct use of a conventional detection method or dry slides. Moreover, the method of the present invention enables shortening of the measurement time for both separation and detection to a significant extent. Thus, the method of the present invention is extremely useful in clinical examinations. Further, in the present invention, the magnetic is very small and exists without being precipitated when it is allowed to stand. Therefore, the method of the present invention is advantageous in that the operation of stirring and uniformizing the magnetic before use is unnecessary. Further, in the present invention, when agglutinate is formed with lipoproteins other than HDL, the agglutinate can be precipitated by magnet within 1 minute, and thus the method of the present invention is suitable for automated process.

1. A method for separating a target component in a biological sample, which comprises the steps of: (1) causing a biological sample to come into contact with independently dispersed magnetic nanoparticles having a particle size of 50 nm or less, which have anionic functional groups on their surfaces, so as to form an agglutinate of the magnetic nanoparticles and biomolecules capable of interacting with the magnetic nanoparticles; and (2) collecting the agglutinate by an external magnetic field.

2. The method according to claim 1, wherein the biomolecules capable of interacting with the magnetic nanoparticles have a size that is the same as or greater than the particle size of the magnetic nanoparticles.

3. The method according to claim 1 or 2, wherein the magnetic nanoparticles are surfaces-modified with a compound represented by the formula R¹—(OCH₂CH₂)ₙ—O-L-X wherein R¹ represents a C1-24 alkyl group, n represents an integer of 1 to 20, L represents a single bond or a C1-10 alkylene group, and X represents a carboxylic acid group, a phosphoric acid group, a sulfonic acid group, or a boric acid group.

4. The method according to claim 1, which comprises forming an agglutinate of lipoproteins other than a specific lipoprotein fraction in a biological sample and magnetic nanoparticles, and collecting the agglutinate by an external magnetic field, so as to separate the specific lipoprotein fraction in the biological saniple.

5. The method according to claim 4, wherein the specific fraction is a high density lipoprotein (HDL).

6. The method according to claim 4 or 5, wherein the specific fraction is separated for the determination of the amount of cholesterol contained in the specific fraction.

7. The method according to claim 6, wherein the biological sample is caused to come into contact with magnetic nanoparticles in the coexistence of an agglutination-promoting agent.

8. The method according to claim 7, wherein a polyanion is used as the agglutination-promoting agent.

9. The method according to claim 8, wherein the polyanion is a polyanion which is selected from phosphotungstic acid, dextrin sulfate, cyclodextrin sulfate, Calixarene, or heparin.

10. The method according to claim 1, wherein the independently dispersed magnetic nanoparticles having a particle size of 50 nm or less are magnetites.

11. A clinical examination method, which comprises the steps of (1) separating a target component in a biological sample by the method according to claim 1, and (2) determining the amount of the thus separated target component.

12. The clinical examination method according to claim 11, wherein the amount of a target component is determined using a dry analytical element.

13. An automatic clinical examination apparatus, which comprises at least (1) a vessel wherein magnetic nanoparticles are caused to come into contact with a biological sample so as to form an agglutinate, (2) a magnetic field generation means for generating a magnetic field for collecting the agglutinate within the vessel, and (3) a dry analytical element for detecting a target component in the biological sample which was separated from the agglutinate.

14. An examination kit for performing the method according to claim 1, which comprises independently dispersed magnetic nanoparticles having a particle size of 50 mm or less which have anionic functional groups on their surfaces.
